(19) United States
(12) Patent Application Publication
(10) Pub. No.: US 2009/0283703 A1
(43) Pub. Date: Nov. 19, 2009

(54) ION BEAM IRRADIATION APPARATUS AND ION BEAM MEASURING METHOD

(75) Inventor: Shigehisa Tamura, Kyoto (JP)

Correspondence Address: OSHA LIANG L.L.P., TWO HOUSTON CENTER, 909 FANNIN, SUITE 3500, HOUSTON, TX, 77010, US

(73) Assignee: NISSIN ION EQUIPMENT CO., LTD., Kyoto (JP)

(21) Appl. No.: 12/468,625

(22) Filed: May 19, 2009

(30) Foreign Application Priority Data
May 19, 2008 (JP) 2008-131115

(51) Int. Cl. A61N 5/00; G21K 1/00

(52) U.S. Cl. 250/492.3; 250/505.1

(57) ABSTRACT

A beam profile monitor is disposed on an orbit of an ion beam, and measures a beam intensity distribution of the ion beam. A pair of beam blocking members are opposed to each other across the ion beam in the x direction, and forms an opening through which the ion beam passes: At least one of the beam blocking members includes a plurality of movable blocking plates disposed without forming a gap in the y direction, and in an independently reciprocable manner in the x direction. A minute opening is formed between the beam blocking members opposed to each other by adjusting the positions of the beam blocking members. From a result of the intensity distribution measurement which is performed by said beam profile monitor on the ion beam passed through the minute opening, the emittance of the ion beam is calculated.

### FIELD OF THE INVENTION

The present disclosure relates to an ion beam irradiation apparatus and ion beam measuring method which can measure an emittance of, for example, a ribbon-like ion beam.

### DESCRIPTION OF RELATED ART

In ion beam irradiation apparatuses which are used in an ion implantation system and the like, in order to suitably control the emitted ion beam, some ion beam irradiation apparatuses have a function of measuring an emittance of the ion beam. In a related-art emittance measurement of this kind, a blocking member having a beam passing hole such as a slit or a small hole is specially disposed, a spreading angle of the ion beam passed through the beam passing hole is measured by a sensor, and the emittance of the ion beam is calculated.

In Japanese Patent Publication No. JP-A-2005-63874, for example, first and second slits are placed on an orbit of an ion beam, the slits are moved perpendicularly with respect to a traveling direction of the ion beam, and currents of ion beams passed respectively through the first and second slits are measured by Faraday cups, thereby allowing the emittance of the ion beam to be calculated. In Japanese Patent Publication JP-A-H06-131999, a blocking plate having a small hole is disposed on an orbit of an ion beam, a fluorescent plate which is caused to emit light by irradiation of an ion beam is disposed behind the blocking plate, and a luminescence image of the fluorescent plate is detected by an area image element to measure the spreading angle of the ion beam passed through the small hole, thereby allowing the emittance of the ion beam to be calculated.

In such related-art ion beam irradiation apparatuses, however, a shape of the beam passing hole and the like are previously set. Therefore, a degree of freedom according to a mode of the emittance measurement is low, and, as described above, a dedicated mechanism for measuring the emittance is required. When the whole system is considered, there are possibilities that a size is enlarged, and that a production cost is increased.

### SUMMARY OF INVENTION

Illustrative aspect of the invention provides an ion beam irradiation apparatus and ion beam measuring method which can be used not only in emittance measurement, but also in other uses such as intensity uniformity of an ion beam, and which, when a whole system including the apparatus is considered, can promote the cost reduction and the simplification.

According to a first aspect of the invention, the ion beam irradiation apparatus is provided with the following configurations (1) to (4):

(1) a beam profile monitor which is disposed on an orbit of the ion beam, and which measures a beam intensity distribution of the ion beam;

(2) a pair of beam blocking members which are opposed to each other across the ion beam in a x direction while being forwardly separated by a constant distance from the beam profile monitor, and which forms an opening through which the ion beam pass, at least one of the beam blocking members including a plurality of movable blocking plates disposed without forming a gap in a y direction as seen in a z direction, and in an independently reciprocable manner in the x direction;

(3) a drive mechanism which reciprocally drives the beam blocking members in the x direction as seen in the z direction; and

(4) a control device which receives a result of the intensity distribution measurement from the beam profile monitor, and which control positions of the beam blocking members via the drive mechanism. More specifically, the control device includes: a minute-opening forming portion which controls positions of the movable blocking plates to form a minute opening between the beam blocking members opposed to each other; and an emittance calculating portion which, from a result of the intensity distribution measurement which is performed by the beam profile monitor on an ion beam passed through the minute opening, calculates an emittance of the ion beam.

In the above description, the z direction is the design traveling direction of the ion beam, the y direction is one direction in a cross-section of the ion beam perpendicular to the z direction, and the x direction is a direction in the cross-section and perpendicular to the y direction.

In the thus configured ion beam irradiation apparatus, the emittance of the ion beam can be measured by using the minute opening formed by the movable blocking plates. The opening is formed by the control of the positions of the plural movable blocking plates. Therefore, for example, the size of the opening can be changed, and not only the one opening but also plural openings can be simultaneously formed, so that the optimum opening mode suited to the emittance measurement can be obtained.

When the size of the opening is further enlarged, the ion beam irradiation apparatus can be used for other purposes other than the emittance measurement. According to a second aspect of the invention, in a measurement of a ribbon-like ion beam in which the dimension of a section of the ion beam in the y direction is larger than that in the x direction, for example, the movable blocking plates may be arranged on the basis of a result of the measurement by the beam profile monitor to control the amount of blocking of the beam so that the beam intensity distribution of the ion beam in the y direction approaches to uniformity.

According to a third aspect of the invention, both of the beam blocking members may include a plurality of movable blocking plates which are disposed without forming a gap in the y direction as seen in the z direction, and in an independently reciprocable manner in the x direction. In this configuration, the degrees of freedom of the shape and position of the opening can be enhanced to make the effects of the invention more remarkable.

According to a fourth aspect of the invention, an ion beam measuring method of the invention uses the beam profile monitor and the pair of beam blocking members, and is characterized in that the method performs the following steps of (1) and (2):

(1) a minute-opening forming step of adjusting positions of the movable blocking plates to form a minute opening between the opposing beam blocking members; and

(2) an emittance calculating step of, from a result of an intensity distribution measurement which is performed by the beam profile monitor on an ion beam that has passed through the minute opening, calculating an emittance of the ion beam.

Other aspects and advantages of the invention will be apparent from the following description, the drawings and the claims.

### BRIEF DESCRIPTION OF THE DRAWINGS

FIG. 1 is a schematic overall view showing the whole of an ion beam irradiation apparatus of an exemplary embodiment of the invention.

FIG. 2 is a perspective view schematically showing beam blocking members in the exemplary embodiment.

FIG. 3 is a functional block diagram of a control device in the exemplary embodiment.

FIG. 4 is a principle diagram illustrating the principle of an emittance measurement in the exemplary embodiment.

FIG. 5 is a principle diagram illustrating the principle of the emittance measurement in the exemplary embodiment.

FIG. 6 is a view showing a state where a plurality of minute openings are formed in the exemplary embodiment, as seen in the z direction.

FIG. 7 is a view showing a state where a uniformity step is performed in the exemplary embodiment, as seen in the z direction.

FIG. 8 is a view schematically showing beam blocking members in another exemplary embodiment of the invention, as seen in the z direction.

FIG. 9 is a schematic perspective view of movable blocking plates in a further exemplary embodiment of the invention.

FIG. 10 is a schematic perspective view of movable blocking plates in a still further exemplary embodiment of the invention.

### DETAILED DESCRIPTION OF EXEMPLARY EMBODIMENTS

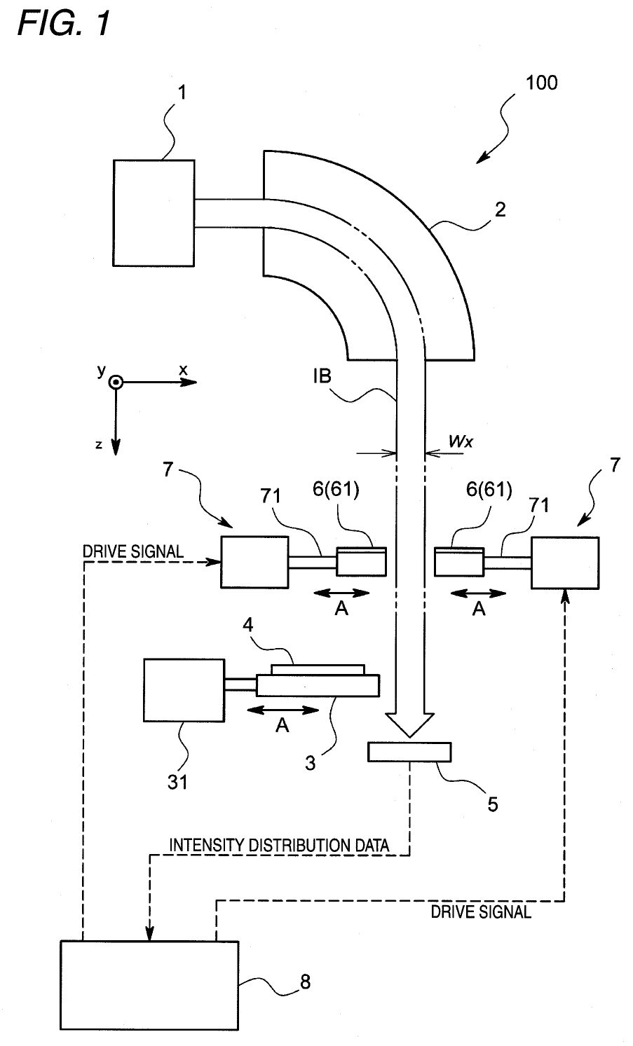

Hereinafter, an exemplary embodiment of the invention will be described with reference to FIGS. 1 to 10.

FIG. 1 shows a schematic overall view of an ion beam irradiation apparatus 100. An ion beam IB extracted from an ion source 1 passes through a mass analyzer 2 to undergo mass separation, and, as required, is accelerated or decelerated. Then the ion beam IB irradiates on a target 4 held by a holder 3 to perform a process such as ion implantation on the target 4. The orbit of the ion beam IB is kept to a vacuum atmosphere. In some cases, the mass analyzer 2 may not be disposed. In the case where ion implantation is performed on the target 4, the apparatus 100 is also called an ion implanting apparatus.

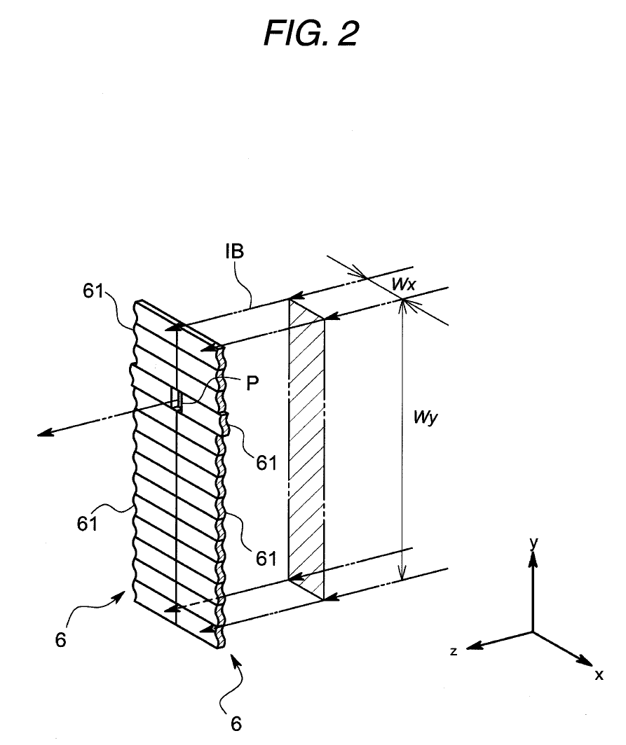

As shown in FIG. 2, the ion beam IB irradiated on the target 4 has a shape in which the dimension W_{y} in the y direction (for example, the longitudinal direction) of a section intersecting with (for example, perpendicular to) the design traveling direction z is larger than the dimension Wₓ in the x direction perpendicular to the y direction. The ion beam IB having such a shape is sometimes called also a ribbon-like, sheet-like, or strip-like ion beam. However, this shape does not mean that the x-direction dimension Wₓ is as thin as paper. For example, the y-direction dimension W_{y} is about 350 to 400 mm, and the x-direction dimension Wₓ is about 80 to 100 mm.

In the exemplary embodiment, the ion beam IB which has not undergone a y-direction scanning process, i.e., the shape itself of the ion beam extracted from the ion source 1 has a ribbon-like shape. Alternatively, as described above, the ion beam IB irradiating on the target 4 may have a ribbon-like shape as result of a y-direction scanning process (for example, parallel scanning) which is performed on the downstream side of the mass analyzer 2.

For example, the target 4 is a semiconductor substrate, a glass substrate, or the like. In the exemplary embodiment, the target 4 is held by the holder 3, and reciprocally driven in a mechanical manner (mechanically scanned) by a target driving device 31 in the x direction as shown by the arrow A. The y-direction dimension W_{y} of the ion beam IB is slightly larger than the dimension of the same direction of the target 4. The combination of this configuration and the above-described reciprocal driving allows the ion beam IB to irradiate on the whole surface of the target 4.

A beam profile monitor 5 is disposed in the vicinity of the target 4 which is at the irradiating position of the ion beam IB, or in the exemplary embodiment in the vicinity of the rear side of the target 4. The beam profile monitor 5 measures the beam intensity distribution (in the embodiment, the current density distribution) in a cross-section of the ion beam IB.

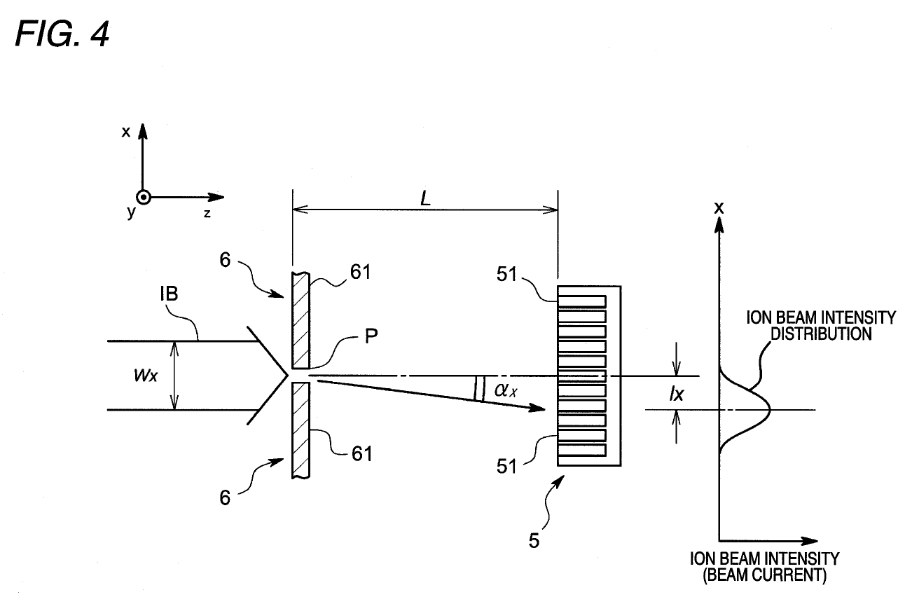

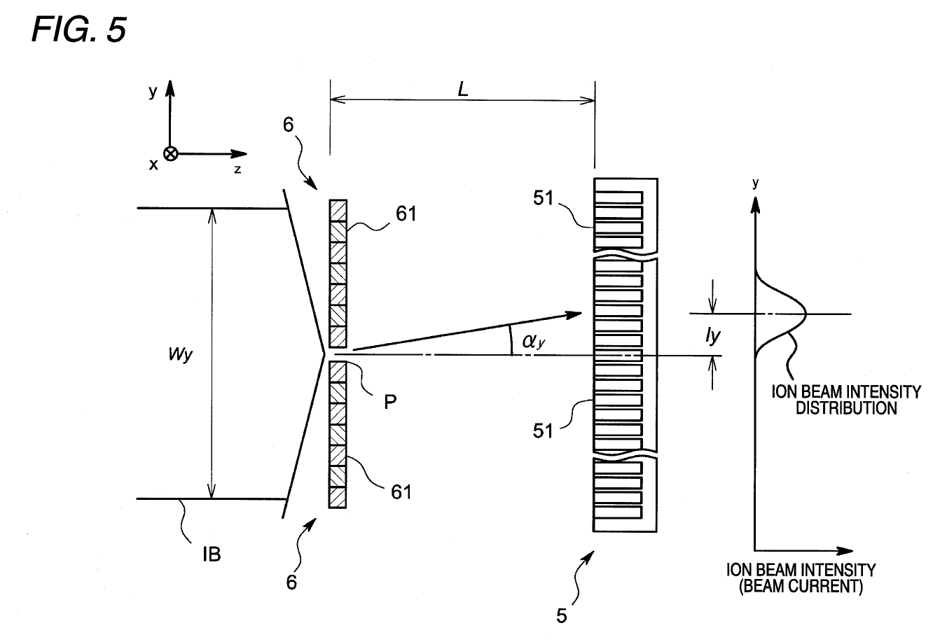

The beam profile monitor 5 is a two-dimensional measuring instrument which measures the beam current density distributions of both the y and x directions of the ion beam IB. In the beam profile monitor 5, for example, many measuring elements (for example, Faraday cups) 51 for measuring the beam current density of the ion beam IB are juxtaposed to each other in the y and x directions (see FIGS. 4 and 5).

During the measurement by the beam profile monitor 5, the target 4 is retracted by the target driving device 31 to a position where the target 4 does not interfere with the beam orbit.

In the exemplary embodiment, as shown in FIGS. 1 and 2, a pair of symmetric beam blocking members 6 which are opposed to each other across the ion beam IB in the x direction are disposed on the upstream side of the position of the target 4. Each of the beam blocking members 6 includes a plurality of movable blocking plates 61 which are juxtaposed in the y direction, and which have a long rectangular shape. Each of the movable blocking plates 61 is in close contact with other movable blocking plates 61 which are adjacent to the movable blocking plates 61 in the y direction, and configured so as to be independently reciprocable in the x direction. The movable blocking plates 61 are not necessary to be in close contact with other movable blocking plates 61 which are adjacent in the y direction, as far as the movable blocking plates 61 are configured so as not to form a gap in the y direction as seen in the z direction.

As shown in FIG. 1, each of the movable blocking plates 61 is reciprocally driven by driving mechanisms 7. The driving mechanisms 7 are disposed respectively for the movable blocking plates 61. For example, each of the driving mechanisms 7 includes a motor (not shown) and a drive rod 71 which is coupled to the motor through a screw feeding structure (not shown). The movable blocking plate 61 is connected to a tip end of the drive rod 71. When the motor is rotated forwardly or reversely, the drive rod 71 and the movable blocking plate 61 are reciprocally moved in the x direction. For example, a rotation angle of the motor from the origin is detected by a position sensor (not shown) such as a rotary encoder, thereby enabling a tip end position of each of the movable blocking plates 61 to be known by a control device 8 which will be described later.

The control device 8 receives data from the beam profile monitor 5, i.e., data of the intensity distribution, and outputs a drive signal to the driving mechanisms 7 to control the driving of the beam blocking members 6 (more specifically, the movable blocking plates 61).

When expressed in terms of hardware, the control device 8 is an electric circuit having a central processing unit (CPU), a memory, an input/output (I/O) channel, an analog to digital (A/D) converter, a digital to analog (D/A) converter, and the like (not shown). Although the control device 8 is integrally shown in FIG. 1, the control device 8 is not necessary to be physically integrated, and may be configured by a plurality of components which are communicably connected to one another.

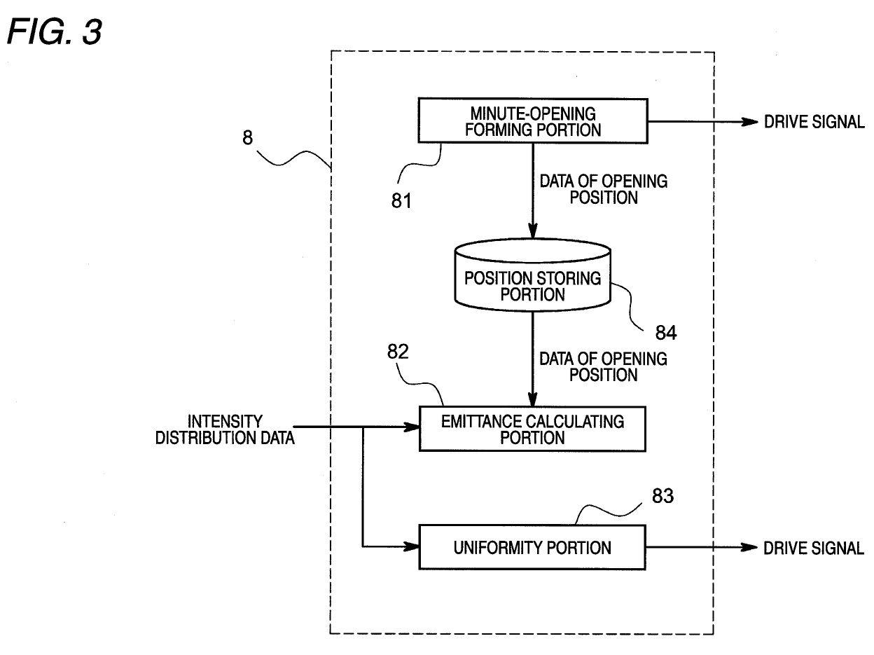

As shown in the functional block diagram of FIG. 3, when the CPU and its peripheral devices cooperate together in accordance with programs which are previously stored in the memory, the control device 8 performs functions of: a minute-opening forming portion 81 which outputs the drive signal to the driving mechanisms 7 to control the positions of the movable blocking plates 61 to form a minute opening P (see FIGS. 2, 4, and 5) between the beam blocking members 6 opposed to each other; an emittance calculating portion 82 which, from a result of the intensity distribution measurement which is performed by the beam profile monitor 5 on the ion beam IB passed through the minute opening P, calculates the emittance of the ion beam IB; etc.

Next, the operation of the thus configured ion beam irradiation apparatus 100 will be described while emphasis is placed particularly on the operation of measuring the emittance.

First, the minute-opening forming portion 81 moves paired ones of the movable blocking plates 61 in, for example, the x direction to form a minute opening between the paired movable blocking plates 61. With respect to all of the other movable blocking plates 61, the other movable blocking plates 61 butt against the opposing movable blocking plates 61 so as not to form a gap between the other movable blocking plates 61 opposed to each other. Between the pair of movable blocking plates 61 and the movable blocking plates 61 which are adjacent to the paired movable blocking plates 61 in the y direction, therefore, one minute opening P is formed as shown in FIG. 2 (minute-opening forming step). On the other hand, from the driving distance of the pair of movable blocking plates 61, the minute-opening forming portion 81 calculates coordinates of the minute opening P in the x and y directions, and stores data of the opening position into a position storing portion 84 which is formed in a threshold region of the memory.

Most of the ribbon-like ion beam IB emitted from the ion source 1 is blocked by the movable blocking plates 61, and only a part of the ion beam which has passed through the minute opening P irradiates on the beam profile monitor 5. As described above, the beam profile monitor 5 is a two-dimensional area sensor for the y and x directions, and therefore outputs the beam intensity distribution in a cross-section of the ion beam IB which has passed through the minute opening P, as intensity distribution data correlated to the x and y coordinates. FIGS. 4 and 5 show a principle of the detection of the beam intensity distribution by the beam profile monitor 5, and a concept of the beam intensity distribution.

Next, the emittance calculating portion 82 receives the intensity distribution data, and obtains the position data of the minute opening P from the position storing portion 84. Based on the intensity distribution data and the position data, the emittance of the ion beam IB at the position of the minute opening P, i.e., the divergence angle αₓ in the x direction and the divergence angle α_{y} in the y direction are calculated (emittance calculating step).

The calculation expressions are as follows:

αₓ=tan⁻¹(1ₓ/L) (1)

α_{y}=tan⁻¹(1_{y}/L) (2)

where L indicates the distance in the z direction between the minute opening P and the beam profile monitor 5, 1ₓ indicates the distance in the x direction between the peak position of the ion beam intensity (current) detected by the beam profile monitor 5 and the minute opening P, and 1_{y} indicates the distance in the y direction between the peak position of the ion beam intensity (current) detected by the beam profile monitor 5 and the minute opening P (see FIGS. 4 and 5).

Therefore, the thus configured ion beam irradiation apparatus 100 of the exemplary embodiment can measure the emittance of the ion beam IB at the position of the minute opening P. When the position of the minute opening P is sequentially changed and the emittance is measured at each of the positions, also the emittances at various positions of the ribbon-like ion beam can be measured accurately and easily.

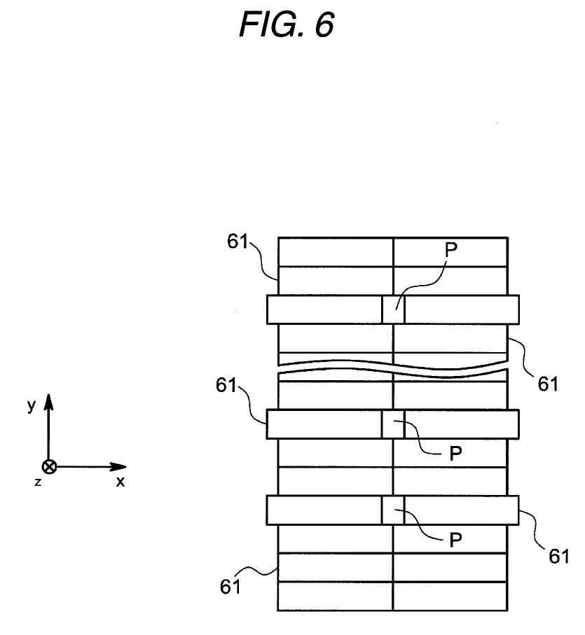

When a plurality of minute openings P are simultaneously formed as shown in FIG. 6, the measurement time can be shortened. The size of the opening P can be freely changed. Therefore, an adequate emittance measurement suited to the mode of the ion beam IB can be flexibly performed.

Even when the ion beam irradiation apparatus 100 has the same hardware, moreover, other functions can be easily installed. In the exemplary embodiment, for example, the function of a uniformity portion 83 shown in FIG. 3 is added to the control device 8 by means of software. The uniformity portion 83 places the movable blocking plates 61 to control an amount of blocking of the beam so that the beam intensity distribution of the ion beam IB in the y direction approaches to uniformity.

Specifically, the uniformity portion 83 first separates all of the movable blocking plates 61 from the opposing movable blocking plates 61 so as to allow substantially most of the ion beam IB to pass in the y direction. Here, for example, a fully open state is set.

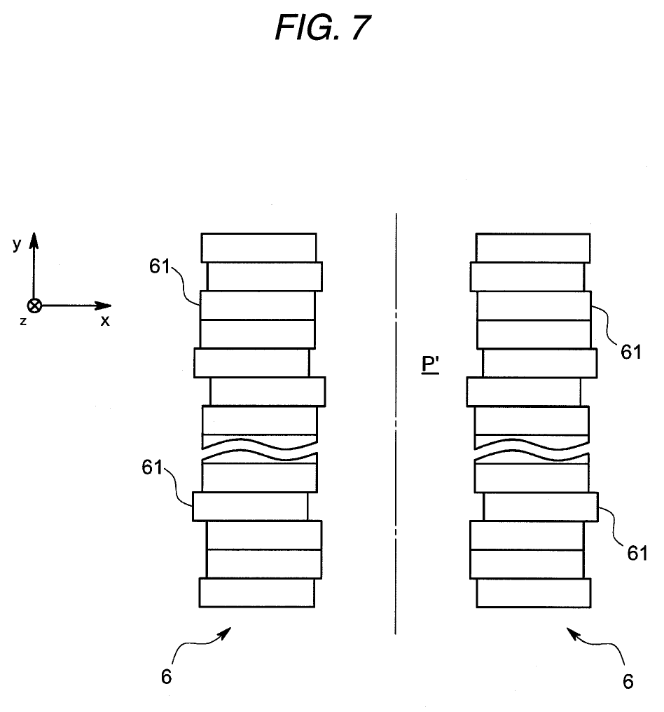

Next, the uniformity portion 83 receives the intensity distribution data from the beam profile monitor 5. In a region where the beam intensity in the y direction is low, the distances between the opposing movable blocking plates 61 are increased, and, in a region where the beam intensity in the y direction is high, the distances between the opposing movable blocking plates 61 are decreased, thereby uniforming the beam intensity distribution in the y direction. In this case, a reference value for determination of the level of the beam intensity is necessary. For example, the value is previously calculated from a total amount of ion implantation on the target. As a result, as shown in FIG. 7, for example, the x-direction width of an opening P′ may be partly different.

The invention is not limited to the above-described embodiment.

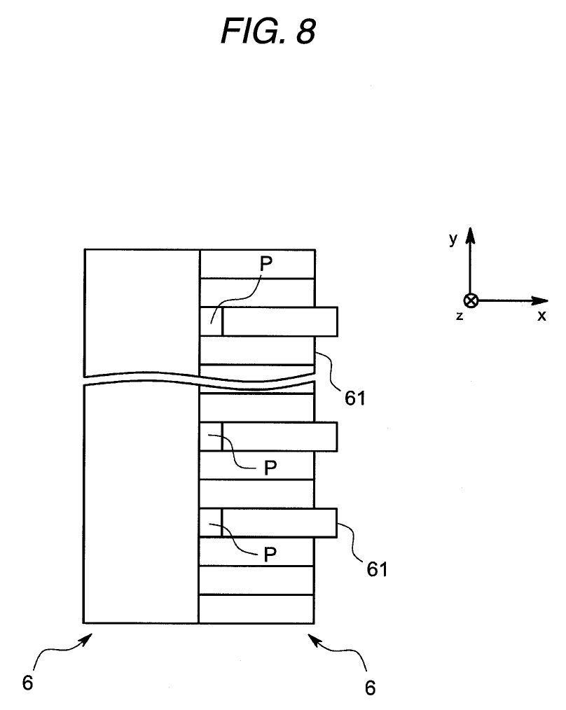

For example, it is not necessary to symmetrically configure the beam blocking members 6. As shown in FIG. 8, for example, only one of the beam blocking members 6 may include a plurality of movable blocking plates 61, and the other beam blocking member 6 may include a single plate. In this example, more preferably, the beam blocking member 6 formed by the single plate may be reciprocated in the x direction by a driving mechanism.

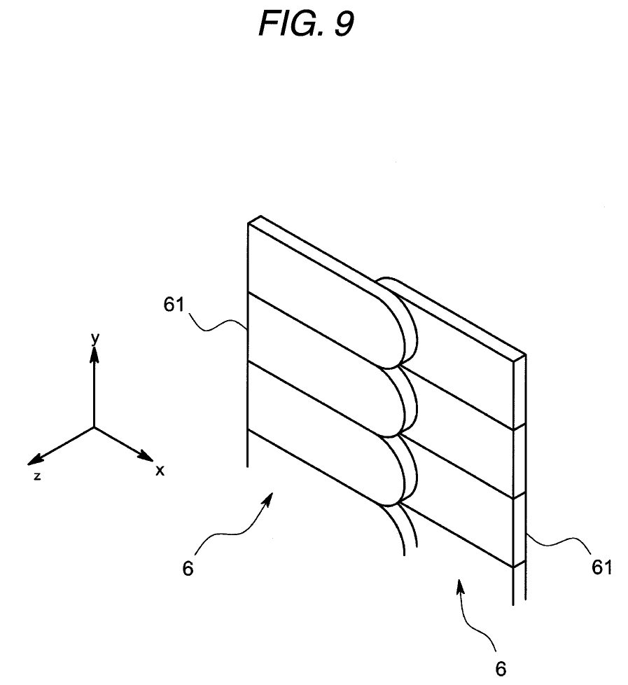

As shown in FIG. 9, alternatively, the movable blocking plates 61 constituting the beam blocking members 6 are placed at different positions in the z direction so that the tip ends of the movable blocking plates 61 can overlap with each other. In the alternative, the tip ends of the movable blocking plates 61 may have various shapes. In FIG. 9, for example, the tip ends have a semicircular shape. The tip ends may have other shapes such as a triangular shape and a recessed circular shape, so that the opening has a shape other than a rectangular shape.

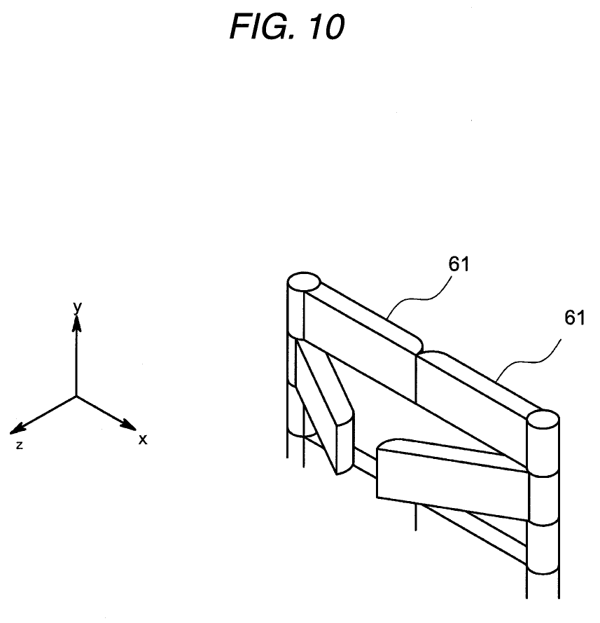

In the specification, the term “reciprocated in the x direction” means that the movable blocking plates are moved in the x direction as seen in the z direction. Therefore, for example, the configuration shown in FIG. 10 may be possible where the movable blocking plates 61 are swingably supported by shafts which are parallel to the y direction, and the movable blocking plates 61 are swung by a motor or the like. By the swinging operation, the tip ends of the movable blocking plates 61 are moved in the x direction as seen in the z direction to form or close an opening.

Alternatively, the beam profile monitor may be configured as a one-dimensional measuring instrument having a linear shape, and a sequential measurement may be performed while the monitor is moved in a direction perpendicular to the linear shape, so that a two-dimensional distribution of the ion beam intensity is measured.

The invention is not limited the above-described embodiment, and it is matter of course that various changes and modifications can be made without departing from the spirit and scope of the invention.

While the present invention has been shown and described with reference to certain exemplary embodiments thereof, it will be understood by those skilled in the art that various changes in form and details may be made therein without departing from the spirit and scope of the invention as defined by the appended claims.

1. An ion beam irradiation apparatus which, in a case where a design traveling direction of an ion beam is set as a z direction, one direction in a cross-section of the ion beam perpendicular to the z direction is set as a y direction, and a direction in the cross-section and perpendicular to the y direction is set as an x direction, said ion beam irradiation apparatus comprising:
a beam profile monitor which is disposed on an orbit of the ion beam, and which measures a beam intensity distribution of the ion beam;
a pair of beam blocking members which are opposed to each other across the ion beam in the x direction while being forwardly separated by a constant distance from said beam profile monitor, and which forms an opening through which the ion beam pass;
a drive mechanism which is connected to said beam blocking member, and which reciprocally drives said beam blocking members in the x direction as seen in the z direction; and
a control device which receives a result of the intensity distribution measurement from said beam profile monitor, and which controls positions of said beam blocking members via said drive mechanism, wherein
at least one of said beam blocking members includes a plurality of movable blocking plates disposed without forming a gap in the y direction as seen in the z direction, and in an independently reciprocable manner in the x direction, and said control device includes:
a minute-opening forming portion which controls positions of said movable blocking plates to form a minute opening between said beam blocking members opposed to each other; and
an emittance calculating portion which, from a result of the intensity distribution measurement which is performed by said beam profile monitor on an ion beam passed through said minute opening, calculates an emittance of the ion beam.

2. An ion beam irradiation apparatus according to claim 1, wherein said apparatus is used for measuring a ribbon-like ion beam in which a dimension of a cross-section of the ion beam in the y direction is larger than a dimension in the x direction, and said control device further includes a uniformity portion which allows a substantially whole amount of the ion beam to pass through in the y direction, and which, on the basis of a result of the measurement by said beam profile monitor, arranges said movable blocking plates to control an amount of blocking of the beam so that the beam intensity distribution of the ion beam in the y direction approaches to uniformity.

3. An ion beam irradiation apparatus according to claim 1, wherein both of said beam blocking members include a plurality of movable blocking plates disposed without forming a gap in the y direction as seen in the z direction, and in an independently reciprocable manner in the x direction.

4. An ion beam measuring method which, in a case where a design traveling direction of an ion beam is set as a z direction, one direction in a cross-section of the ion beam perpendicular to the z direction is set as a y direction, and a direction in the cross-section and perpendicular to the y direction is set as an x direction, said ion beam measuring method uses:
a beam profile monitor which is disposed on an orbit of the ion beam, and which measures a beam intensity distribution of the ion beam; and
a pair of beam blocking members which are opposed to each other across the ion beam in the x direction while being forwardly separated by a constant distance from said beam profile monitor, and which forms an opening through which the ion beam pass,
at least one of said beam blocking members includes a plurality of movable blocking plates disposed without forming a gap in the y direction as seen in the z direction, and in an independently reciprocable manner in the x direction, and
said ion beam measuring method comprising:
a minute-opening forming step of adjusting positions of said movable blocking plates to form a minute opening between said beam blocking members opposed to each other; and
an emittance calculating step of, from a result of an intensity distribution measurement which is performed by said beam profile monitor on an ion beam passed through said minute opening, calculating an emittance of the ion beam.

5. An ion beam irradiation apparatus according to claim 2, wherein both of said beam blocking members include a plurality of movable blocking plates disposed without forming a gap in the y direction as seen in the z direction, and in an independently reciprocable manner in the x direction
